(19) United States
(12) Patent Application Publication
(10) Pub. No.: US 2025/0352045 A1
(43) Pub. Date: Nov. 20, 2025

(54) MEDICAL SYSTEMS AND DEVICES FOR CONTROLLING A DIRECTION OF FLUID FLOW

(71) Applicant: Boston Scientific Scimed, Inc., Maple Grove, MN (US)

(72) Inventors: Angelo Frank SZYCHOWSKI, Mendocino, CA (US); Jeffrey A. MEGANCK, North Grafton, MA (US)

(73) Assignee: Boston Scientific Scimed, Inc., Maple Grove, MN (US)

(21) Appl. No.: 19/206,853

(22) Filed: May 13, 2025

Related U.S. Application Data
(60) Provisional application No. 63/647,209, filed on May 14, 2024.

(51) Int. Cl. A61B 1/015; A61B 1/00

(52) U.S. Cl. CPC A61B 1/015; A61B 1/00105

(57) ABSTRACT

Medical devices are described including a medical device that includes a casing assembly with a first portion movably coupled to a second portion, a first fluidics channel, a second fluidics channel, a third fluidics channel, and a fourth fluidics channel. Moving the first portion relative to the second portion may transition the medical device between a first and a second configuration. In the first configuration, the first and second fluidics channels may be fluidly connected, and the third and fourth fluidics channels may be fluidly connected. In the second configuration, the first and third fluidics channels may be fluidly connected, and the second and fourth fluidics channels may be fluidly connected.

### CROSS-REFERENCE TO RELATED APPLICATIONS

This application claims the benefit of priority to U.S. Provisional Application No. 63/647,209, filed on May 14, 2024, which is incorporated by reference herein in its entirety.

### TECHNICAL FIELD

Various aspects of this disclosure relate generally to medical systems and devices for controlling a direction of fluid flow. In particular, aspects of this disclosure relate to systems and devices for controlling fluid flow to and/or from a body lumen.

### BACKGROUND

During medical procedures, such as urological procedures, an operator may insert a medical device into a patient, and guide that medical device through tortuous anatomy for positioning the device at a target treatment site in the body. For example, an operator may insert a ureteroscope into urinary passages to diagnose and/or treat the urinary tract. During a procedure, particulates may be generated, thus obscuring a visualization field of the medical device. To remove the particulates, aspiration techniques may be utilized. However, the particulates may create blocks or clogs within associated fluidics channels, thus presenting procedural challenges.

### SUMMARY

Each of the aspects disclosed herein may include one or more aspects of the features described in connection with any of the other disclosed aspects.

The present disclosure includes a medical device comprising a first portion movably coupled to a second portion, a first fluidics channel extending from a first end of the casing assembly that includes the first portion, a second fluidics channel extending from a second end of the casing assembly that includes the second portion, a third fluidics channel extending from the second end of the casing assembly, and a fourth fluidics channel extending from the first end of the casing assembly. Moving the first portion relative to the second portion may transition the medical device between a first configuration and a second configuration. In the first configuration, the first fluidics channel and the second fluidics channel may be fluidly connected, and the third fluidics channel and the fourth fluidics channel may be fluidly connected. In the second configuration, the first fluidics channel and the third fluidics channel may be fluidly connected, and the second fluidics channel and the fourth fluidics channel may be fluidly connected.

Any of the devices disclosed herein may include any of the following features, additionally or alternatively, in any combination. In the first configuration, the first and second fluidics channels may be in communication and configured to provide fluid flow in a first direction through the casing assembly, and the third and fourth fluidics channels may be in communication and configured to provide fluid flow in a second direction through the casing assembly, opposite the first direction.

The first portion may define a first lumen and a second lumen, and the second portion may define a third lumen and a fourth lumen. In the first configuration, the first lumen may be fluidly connected to the third lumen, and the second lumen may be fluidly connected to the fourth lumen. In the second configuration, the first lumen may be fluidly connected to the fourth lumen, and the second lumen may be fluidly connected to the third lumen. The first portion may be rotatable relative to the second portion.

The casing assembly may include a manifold disposed between the first portion and the second portion. The manifold may include a first lumen, a second lumen, a third lumen, and a fourth lumen extending therethrough. The second lumen of the manifold and the fourth lumen of the manifold may be transverse to a longitudinal axis of the manifold. The first lumen of the manifold and the second lumen of the manifold may be parallel to the longitudinal axis of the manifold. In the first configuration, the first fluidics channel and the second fluidics channel may be fluidly connected via the first lumen of the manifold, and the third fluidics channel and fourth fluidics channel may be fluidly connected via the second lumen of the manifold. In the second configuration, the first fluidics channel and the third fluidics channel may be fluidly connected via the third lumen of the manifold, and the second fluidics channel and the fourth fluidics channel may be fluidly connected via the fourth lumen of the manifold. The casing assembly may include an actuator movable within a window formed by the first portion and the second portion of the casing assembly to move the medical device between the first configuration and the second configuration.

The medical device may include a plurality of sealing members configured to provide a fluid-tight seal around each respective first, second, third, and fourth fluidics channels. One of the first portion or the second portion may include an indexing mechanism. The indexing mechanism may include a projection receivable within an aperture. One of the first portion or the second portion may include the projection and the other of the first portion or the second portion may include the aperture.

The first fluidics channel and the fourth fluidics channel may be configured to be fluidly coupled to a scope. The casing assembly may include a fastener configured to couple the medical device to a scope.

In another example according to the present disclosure, the medical device may include a casing assembly, a first fluidics channel extending from a first end of the casing assembly, a second fluidics channel extending from a second end of the casing assembly, a third fluidics channel extending from the second end of the casing assembly, and a fourth fluidics channel extending from the first end of the casing assembly. The casing assembly may include an actuator to transition the medical device between a first configuration and a second configuration. In the first configuration, the first fluidics channel and the second fluidics channel may be fluidly connected. The third fluidics channel and the fourth fluidics channel may be fluidly connected. In the second configuration the first fluidics channel and the third fluidics channel are fluidly connected, and the second fluidics channel and the fourth fluidics channel may be fluidly connected. Optionally, the casing assembly may include a plurality of Y-connectors. Additionally or alternatively, t\The actuator may be movable within a window of the casing assembly to move the medical device between the first configuration and the second configuration.

In another example according to the present disclosure, the medical device may include a casing assembly including a first portion, a second portion, and a manifold between the first portion and the second portion, a first fluidics channel may extend from a first end of the casing assembly that includes the first portion, a second fluidics channel may extend from a second end of the casing assembly that includes the second portion, a third fluidics channel may extend from the second end of the casing assembly, and a fourth fluidics channel may extend from the first end of the casing assembly. The manifold may include a first lumen, a second lumen, a third lumen, and a fourth lumen extending therethrough. In a first configuration of the medical device, the first fluidics channel and the second fluidics channel may be fluidly connected via the first lumen, and the third fluidics channel and the fourth fluidics channel may be fluidly connected via the second lumen. In a second configuration of the medical device, the first fluidics channel and the third fluidics channel may be fluidly connected via the third lumen, and the second fluidics channel and the fourth fluidics channel may be fluidly connected via the fourth lumen. Additionally or alternatively, the casing assembly may include an actuator configured to transition the medical device between the first configuration and the second configuration.

### BRIEF DESCRIPTION OF DRAWINGS

The accompanying drawings, which are incorporated in and constitute a part of this application, illustrate exemplary aspects of this disclosure and together with the description, serve to explain the principles of the present disclosure.

FIG. 1 illustrates an exemplary schematic of a medical system, in accordance with some aspects of the present disclosure.

FIGS. 2A and 2B illustrate exemplary schematics of a fluid delivery module of the medical system of FIG. 1 in a first configuration (FIG. 2A) and a second configuration (FIG. 2B), in accordance with some aspects of the present disclosure.

FIG. 3 illustrates an exemplary medical device of the medical system of FIG. 1, in accordance with some aspects of the present disclosure.

FIGS. 4A and 4B illustrate an exemplary fluid delivery module, in accordance with some aspects of the present disclosure.

FIGS. 4C and 4D illustrate cross-sectional views of fluid flow within the fluid delivery module of FIGS. 4A and 4B in a first configuration (FIG. 4C) and in a second configuration (FIG. 4D), in accordance with some aspects of the present disclosure.

FIGS. 5A-5E illustrate various views of an alternative exemplary fluid delivery module, in accordance with some aspects of the present disclosure.

FIGS. 6A-6C illustrate various views of a further alternative exemplary fluid delivery module, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Throughout various figures, “P” and “D” may be used to illustrate proximal and distal directions, respectively.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although ureteroscopes are referenced herein for illustration purposes, it will be appreciated that the disclosure encompasses any suitable medical device configured to allow an operator to access and view internal body anatomy of a subject (e.g., patient) and/or to deliver medical instruments or accessory devices, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, lungs, the urinary tract, or gastrointestinal tract. It will be appreciated that, unless otherwise specified, endoscopes, duodenoscopes, gastroscopes, endoscopic ultrasonography (“EUS”) scopes, colonoscopes, bronchoscopes, laparoscopes, cystoscopes, aspiration scopes, sheaths, catheters, or any other suitable delivery device or medical device may be used in connection with the features described herein.

Features of the medical systems herein may improve visualization within tortuous body anatomy (e.g., the urinary tract) and/or the delivery or removal of fluids within a subject. According to some aspects of the present disclosure, the medical system may include a medical device, e.g., a scope (e.g., a ureteroscope) and a control unit. While the discussion herein uses the term scope, it is understood that the present disclosure includes any type of medical device configured to control fluid flow, e.g., with a fluidics module. The scope (or other medical device) may include an insertion portion (e.g., a shaft) configured for insertion into a subject. A distal end of the insertion portion may include one or more imaging devices and/or light sources. The control unit may include a monitor or a screen, for example, to display information transmitted from the scope. The medical system may further include a fluid delivery module in fluid connection with the scope and each of a vacuum source and a fluid source. The fluid delivery module may control a direction of fluid flow between the vacuum source and/or fluid source and the scope.

Reference will now be made in detail to examples of the present disclosure described above and illustrated in the accompanying drawings. Wherever possible, the same reference numbers will be used throughout the drawings to refer to the same or like parts.

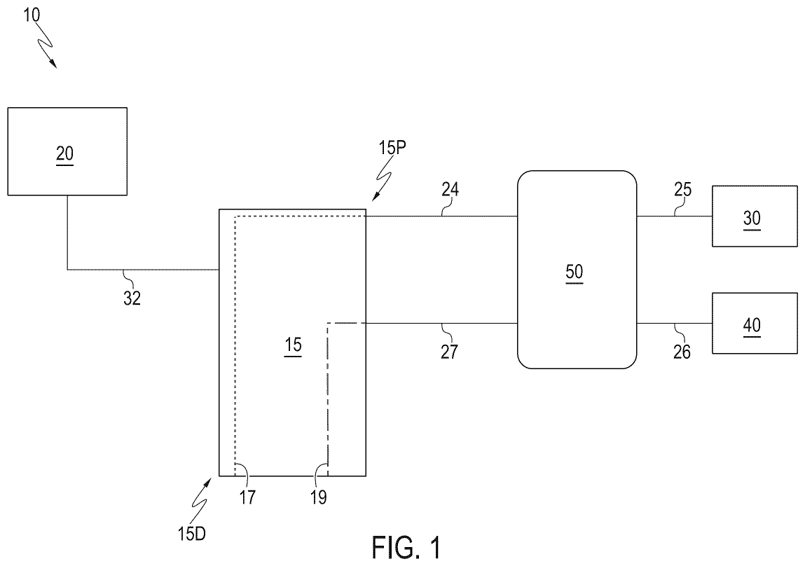

FIG. 1 illustrates a schematic of an exemplary medical system 10 comprising a scope 15 (or any other medical device), a control unit 20, a negative pressure (e.g., vacuum) source 30, a fluid source 40, and a fluidics module 50. Scope 15 may include two or more lumens in fluid connection with fluidics module 50 at a proximal end 15P of scope 15. For example, fluidics module 50 may be fluidly connected to a first lumen 17 via a first fluidics channel 24 extending therebetween. Vacuum source 30 may be fluidly connected to fluidics module 50 via a second fluidics channel 25 extending therebetween. Fluidics module 50 may also be fluidly connected to fluid source 40 via a third fluidics channel 26 extending therebetween. Fluidics module 50 may be fluidly connected to a second lumen 19 of scope 15 via a fourth fluidics channel 27 extending therebetween. Described in further detail below, fluidics module 50 may be configured to control a direction of fluid flowing through each of first lumen 17 and second lumen 19 of scope 15. It will be appreciated that the “fluidics channel(s)” discussed herein may be any flexible, semi-flexible, or rigid channel, conduit, tube, cannula, pipe, or any other device or component configured to carry a fluid.

Lumens 17, 19 may have respective openings at a distal end 15D of scope 15. Distal end 15D may also include at least one of an imaging device (e.g., a camera) and/or a light source (e.g., a light emitting diode). Images captured via the imaging device of scope 15 may be transmitted to control unit 20. For example, imaging data may be transmitted wirelessly and/or via a connector 32. Connector 32 may extend from scope 15 and be removably connected to control unit 20. Control unit 20 may include an internal display and/or be connected to an external display to display the images captured via the imaging device of scope 15.

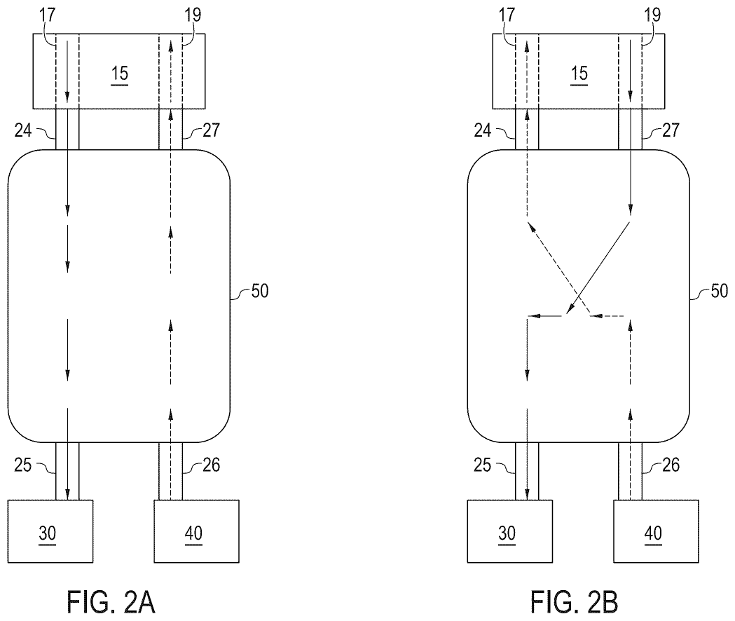

FIGS. 2A and 2B each illustrate a portion of system 10, shown in FIG. 1. For example, FIG. 2A illustrates a schematic of fluidics module 50 in a first configuration, and FIG. 2B illustrates a schematic of fluidics module 50 in a second configuration. In the first configuration of fluidics module 50 (FIG. 2A), fluid may flow in a first direction within first lumen 17 of scope 15, e.g., towards fluidics module 50. For example, fluid may flow from lumen 17 and into fluidics module 50 (e.g., via first fluidics channel 24 extending between scope 15 and fluidics module 50). The fluid may flow from fluidics module 50 into a reservoir of vacuum source 30 (e.g., via second fluidics channel 25 extending between fluidics module 50 and vacuum source 30). In these aspects, first lumen 17 may be in fluid communication with vacuum source 30.

In the first configuration of fluidics module 50, fluid may also flow in a second direction within second lumen 19, e.g., away from fluidics module 50. For example, the fluid may flow from fluid source 40, into fluidics module 50 (e.g., via third fluidics channel 26 extending between fluidics module 50 and fluid source 40). The fluid may flow then through fluidics module 50 and into second lumen 19 of scope 15 (e.g., via fourth fluidics channel 27 extending between scope 15 and fluidics module 50). In these aspects second lumen 19 may be in fluid communication with fluid source 40.

In a second configuration of fluidics module 50 (FIG. 2B), a direction of fluid flow may be reversed in each of first lumen 17 and second lumen 19 of scope 15. For example, in the second configuration, first lumen 17 may be in fluid communication with fluid source 40, and second lumen 19 may be in fluid communication with vacuum source 30. Thus, a direction of fluid flow within each of first lumen 17 and second lumen 19 may be reversed. The direction of fluid flow within second fluidics channel 25 and third fluidics channel 26 may remain the same as in the first configuration. For example, in the second configuration, the fluid may flow from second lumen 19 of scope 15 into fluidics module 50 (e.g., via fourth fluidics channel 27). The fluid may then flow into a reservoir of vacuum source 30 (e.g., via second fluidics channel 25). The fluid may also flow from fluid source 40, into fluidics module 50 (e.g., via third fluidics channel 26), and distally into first lumen 17 of scope 15 (e.g., via first fluidics channel 24). As will be explained in further detail below, fluidics module 50 may be configured to switch the direction of fluid within first lumen 17 and second lumen 19 of scope 15 in a number of ways.

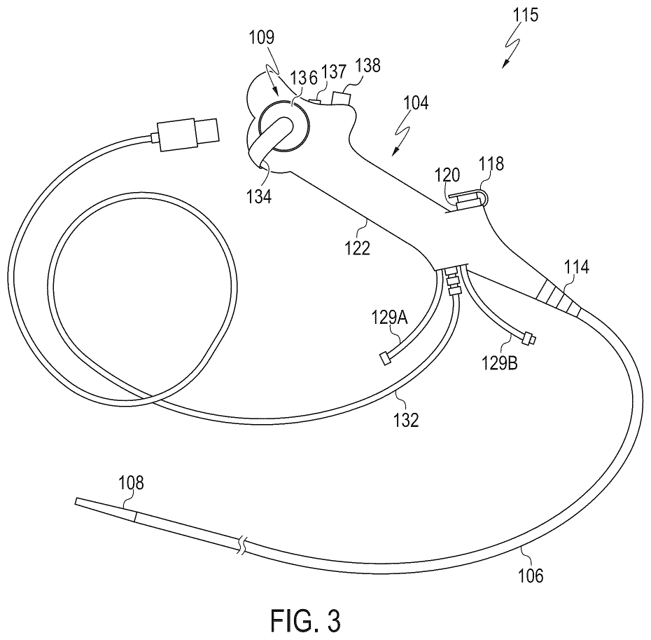

FIG. 3 illustrates an exemplary medical device, e.g., scope 115 (e.g., a ureteroscope) useful in a system such as system 10 of FIG. 1. Scope 115 may have a handle 104 and a shaft 106 extending distally from handle 104. Shaft 106 may be configured for insertion into a subject. A stress relief portion 114 may bridge handle 104 and shaft 106. Shaft 106 may be at least partially flexible to facilitate navigation of shaft 106 through tortuous anatomical passages in the body. Shaft 106 may include a distal end portion that terminates at a distal tip 108. Distal tip 108 may include an imaging device (e.g., camera, imager, etc.), one or more light sources (e.g., LEDs, fiber optics, etc.), and one or more openings in communication with respective lumen(s) extending through shaft 106. For example, a lumen of shaft 106 may extend to a port 120 of handle 104. In some examples, distal tip 108 may include openings for first lumen 17 and second lumen 19, described above. In some examples, shaft 106 may include three lumens, e.g., first lumen 17, second lumen 19, and a third lumen in communication with port 120. An operator (e.g., a user) may remove a cap or seal 118 from port 120, and may insert a medical instrument or other device into port 120 and may extend the medical instrument or other device distally through the corresponding lumen of scope 115. Handle 104 may include a grip portion 122, which may allow an operator to grasp handle 104 during a medical procedure.

Optionally handle 104 may include adapters in communication with first lumen 17 and second lumen 19 to facilitate connection to a fluid source or vacuum source. For example, as shown in FIG. 3, handle 104 may include a first fluid connector 129A and a second fluid connector 129B. Fluid connectors 129A, 129B may be in fluid communication with one or more lumens extending through shaft 106 (e.g., first lumen 17 and/or second lumen 19). For example, first fluidics channel 24 may be removably connected to first fluid connector 129A, thus permitting fluid flow between first lumen 17 and fluidics module 50. Fourth fluidics channel 27 may be removably connected to second fluid connector 129B, thus permitting fluid flow between second lumen 19 and fluidics module 50. Fluid connectors 129A, 129B may extend outward (e.g., away from a longitudinal axis of handle 104), similar to how a connector 132 (e.g., an umbilicus) extends from handle 104. Fluid connectors 129A, 129B may extend outward from any portion of handle 104.

Handle 104 may include an actuation mechanism 109 including one or more actuators, e.g., a lever 134 and a wheel 136 (e.g., a cam wheel). The actuator(s) may control or otherwise facilitate articulation, steering, and/or deflection of shaft 106 and distal tip 108. For example, the actuator(s) may provide for 180-degree articulation in one or more directions. Handle 104 may also include one or more actuators to control electronic components of scope 115. For example handle 104 may include a button 137 for image capture (e.g., configured to capture video and/or still images using the imaging device at distal tip 108). Additionally, in some aspects, handle 104 may include a valve 138 to control suction, e.g., to provide suction of fluid (e.g., gas and/or liquid) through scope 115.

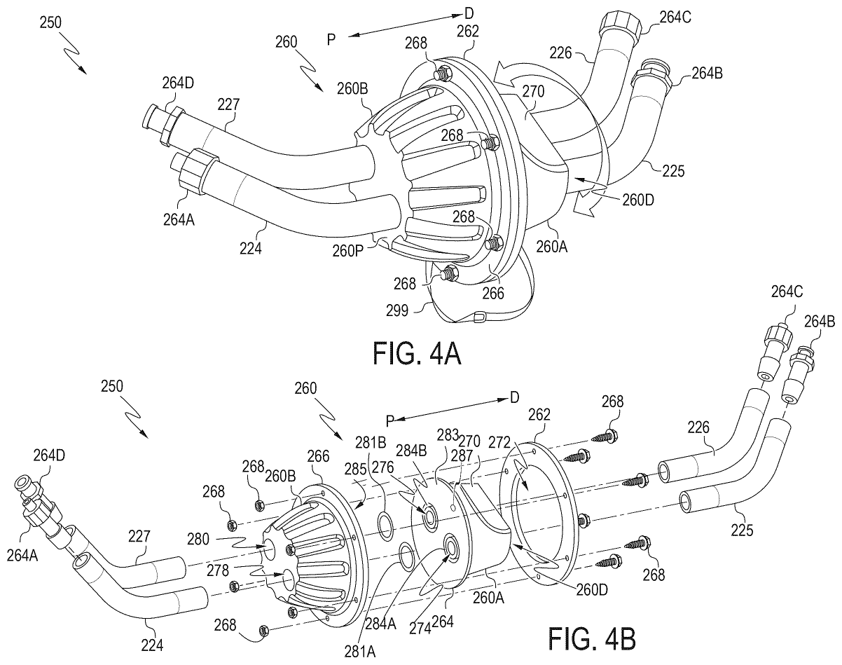

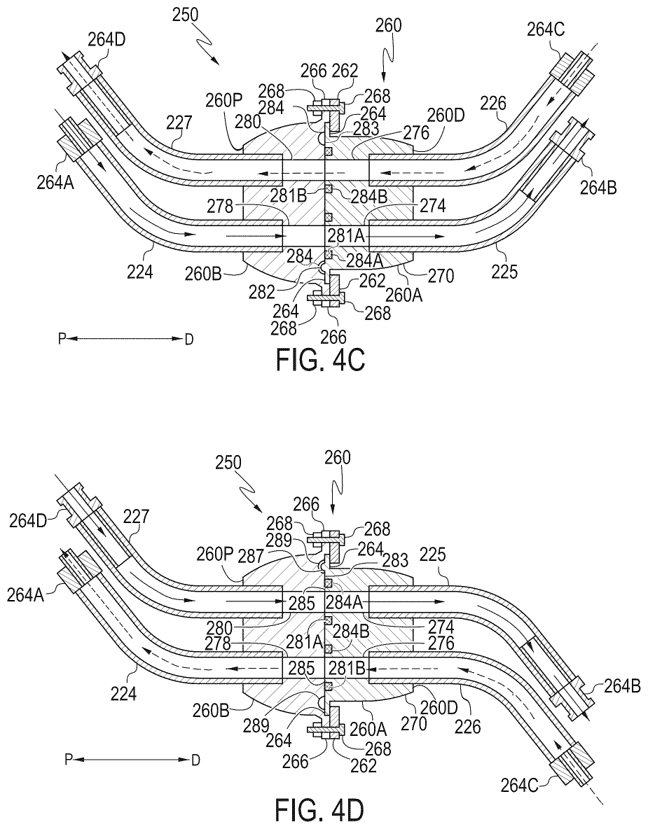

FIGS. 4A-4D illustrate various views of an exemplary fluidics module 250. FIG. 4A illustrates a perspective view of fluidics module 250; FIG. 4B illustrates an exploded view of fluidics module 250; FIG. 4C illustrates a cross-sectional view of fluidics module 250 in a first configuration; and FIG. 4D illustrates a cross-sectional view of fluidics module 250 in a second configuration. Fluidics module 250 may be used with medical system 10 of FIG. 1. In aspects, fluidics module 250 may be used in conjunction with scope 115 of FIG. 3. Fluidics module 250 may be configured to reverse the flow of fluid within lumens of a connected medical device (e.g., first lumen 17 and second lumen 19 of scope 15, shown in FIGS. 1 and 2A-2B).

Fluidics module 250 may include a housing, e.g., casing assembly 260. Casing assembly 260 may have a first casing portion 260A (also referred to herein as a first portion 260A of casing assembly 260) and a second casing portion 260B (also referred to herein as a second portion 260B of casing assembly 260) with fluidic connections to provide a source of fluid and/or vacuum to a medical device. For example, FIGS. 4A-4C show an example with four fluidics channels (e.g., a first fluidics channel 224, a second fluidics channel 225, a third fluidics channel 226, and a fourth fluidics channel 227) extending from casing assembly 260. For example, first fluidics channel 224 and fourth fluidics channel 227 may extend from a first end of casing assembly 260, and second fluidics channel 225 and third fluidics channel 226 may extend from a second end of casing assembly 260 opposite the second end. In some aspects, the first end may be a proximal end in use that extends toward an attached medical device, and the second end may be a distal end in use that extends toward an attached fluid source. Thus, for example, first fluidics channel 224 and fourth fluidics channel 227 may extend proximally from a proximal wall 260P of first casing portion 260A, and second fluidics channel 225 and third fluidics channel 226 may extend distally from a distal wall 260D of second casing portion 260B. While the discussion herein refers to proximal and distal, e.g., relative to a subject in use during a medical procedure, it is understood that the proximal end generally refers to a first end, and the distal end refers to a second end opposite the first end.

Shown more clearly in FIGS. 4C and 4D, first casing portion 260A may include a first lumen 274 and a second lumen 276 extending therethrough. For example, respective openings of first lumen 274 and second lumen 276 may be disposed on or in second (e.g., distal) wall 260D of first casing portion 260A, and respective openings of first lumen 274 and second lumen 276 may be disposed on or in a first (e.g., proximal) wall or face 283 of first casing portion 260A. First lumen 274 of first casing portion 260A may be sized and shaped to at least partially receive a portion of or otherwise be in fluid communication with second fluidics channel 225, and second lumen 276 of first casing portion 260A may be sized and shaped to at least partially receive a portion of or otherwise be in fluid communication with fourth fluidics channel 227.

Second casing portion 260B may similarly include a first lumen 278 and a second lumen 280 extending therethrough. For example, respective openings of first lumen 278 and second lumen 280 of second casing portion 260B may be disposed on or in a wall or face 285 of second casing portion 260B, and respective openings of first lumen 278 and second lumen 280 may be disposed on or in a wall 260P of second casing portion 260B. First lumen 278 of second casing portion 260B may be sized and shaped to at least partially receive a portion of or otherwise be in fluid communication with first fluidics channel 224, and second lumen 280 of second casing portion 260B may be sized and shaped to at least partially receive a portion of or otherwise be in fluid communication with third fluidics channel 226.

According to some examples of the present disclosure, lumens 274, 276, 278, 280 may be parallel to one another. The medical devices herein may include features to facilitate a fluid seal, e.g., against other components of the medical device and/or against the surrounding environment. For example, a first sealing member 281A (e.g., an O-ring or similar) may be disposed in a second groove 284A that extends around the proximal opening of first lumen 274, and a second sealing member 281B (e.g., O-ring or similar) may be disposed in a second groove 284B that extends around the proximal opening of second lumen 276. Each of first groove 283A and second groove 283B may be disposed on proximal face 283 of first casing portion 260A. In some aspects, the sealing members are disposed around the respective lumens without the need for grooves (e.g., the sealing members being elastic and capable of forming a seal without a groove. Sealing members 281A, 281B may be configured to create a fluid-tight seal (e.g., liquid-tight seal) relative to the surrounding environment and/or between the lumens of first casing portion 260A and second casing portion 260B. For example, in the first configuration (see, e.g., FIG. 4C), first lumen 274 of first casing portion 260A may be aligned and in fluid communication with first lumen 278 of second casing portion 260B, and first sealing member 281A may be disposed between the lumens of each casing portion. In the first configuration, second lumen 276 of first casing portion 260A may also be aligned and in fluid communication with second lumen 280 of second casing portion 260B, and second sealing member 281B may be disposed between the lumens of each casing portion.

First casing portion 260A and second casing portion 260B may be coupled together, e.g., such that proximal face 283 of first casing portion 260A abuts distal face 285 of second casing portion 260B. A plate 262 may be placed over a flange 264 of first casing portion 260A. Plate 262 may be secured to a flange 266 of second casing portion 260B via fastening elements, e.g., a plurality of mechanical fasteners 268 (e.g., nuts and bolts, screws, welds, rivets, etc.). In some aspects, plate 262 may be secured to second casing portion 260B using an adhesive such as an epoxy. A grip 270 of first casing portion 260A may extend through an opening 272 of plate 262. In these aspects, plate 262 may secure first casing portion 260A to second casing portion 260B, yet still permit rotation of first and second casing portions 260A, 260B relative to one another. In aspects, first casing portion 260A and second casing portion 260B may be rotatable relative to one another, for example, to transition fluidics module 250 from the first configuration (e.g., FIG. 4C) to the second configuration (e.g., FIG. 4D).

An end of first fluidics channel 224 may be fluidly connected to first lumen 17 (see, e.g., FIGS. 1, 2A, 2B) of scope 15. The distal end of first fluidics channel 224 may include a first coupler 264A. First coupler 264A may facilitate a secure and fluid-tight connection to a first fluid connector of an attached scope (e.g., first fluid connector 127A of scope 115; see FIG. 3). An end of second fluidics channel 225 may include a second coupler 264B. Second coupler 264B may facilitate a secure and fluid-tight connection to a vacuum source (e.g., vacuum source 30 of FIGS. 1, 2A, 2B). An end of third fluidics channel 226 may include a third coupler 264C, which may facilitate a secure and fluid-tight connection to a fluid source (e.g., fluid source 40 of FIGS. 1, 2A, 2B). An end of fourth fluidics channel 227 may include a fourth coupler 264D. Fourth coupler 264D may facilitate a secure and fluid-tight connection to a second connector of an attached scope (e.g., a connector of scope 15 of FIGS. 1, 2A, 2B or second fluid connector 127B of scope 115 of FIG. 3).

In aspects, first coupler 264A and fourth coupler 264D may include different coupling mechanisms, for example, to prevent inadvertently coupling first fluidics channel 224 to the second fluid connector and fourth fluidics channel 227 to the first fluid connector. For example, first coupler 264A may be a female connector and fourth coupler 264D may be a male connector. Similarly, second coupler 264B and third coupler 264C may be different, for example to prevent inadvertently coupling second fluidics channel 225 to the fluid source and third fluidics channel 226 to the vacuum source. For example, second coupler 264B may be a female connector and third coupler 264C may be a male connector.

Referring to FIG. 4C, in the first configuration, fluid may flow in a first direction (e.g., proximally) within first fluidics channel. For example, fluid may flow proximally within a lumen of a scope (e.g., within first lumen 17 of scope 15 of FIGS. 1, 2A, 2B or a first lumen of scope 115 of FIG. 3) and through first fluidics channel 224 of casing assembly 260. The fluid may flow through first fluidics channel 224 and into first lumen 278 of second casing portion 260B. The fluid may flow distally through lumen 280 and through first lumen 274 of first casing portion 260A. The fluid may then flow from first lumen 274 of first casing portion 260A and proximally through second fluidics channel 225 to an attached vacuum source (e.g., vacuum source 30). In the first configuration, the fluid may also from fluid source 40, through third fluidics channel 226 and into second lumen 276 of first casing portion 260A. The fluid may flow from second lumen 276 of first casing portion 260A, through second lumen 280 of second casing portion 260B, and through fourth fluidics channel 227. The fluid may flow from fourth fluidics channel 227 and into or through a second lumen of a connected scope (e.g., second lumen 19 of scope 15 shown in FIGS. 1, 2A).

In the second configuration (FIG. 4D), the fluid connections between the lumens of first casing portion 260A and second casing portion 260B may be switched. For example, in the second configuration, first lumen 274 of first casing portion 260A may be in fluid communication with second lumen 280 of second casing portion 260B. First sealing member 281A may be disposed between first lumen 274 and second lumen 280. Second lumen 276 of first casing portion 260A may also be in fluid communication with first lumen 278 of second casing portion 260B. Second sealing member 281B may be disposed between second lumen 276 and first lumen 278. In these aspects, a direction of fluid flowing within each of first fluidics channel 224 and fourth fluidics channel 227, and, thus, within first lumen 17 and second lumen 19 of scope 15 (FIGS. 1, 2B), may be reversed. For example, in the second configuration, fluid may flow distally from the second lumen of an attached scope (e.g., lumen 19 of scope 15), through fourth fluidics channel 227, into second lumen 280 of second casing portion 260B. The fluid may continue to flow from second lumen 280 of second casing portion 260B and through first lumen 274 of first casing portion 260A. The fluid may flow from first lumen 274A of first casing portion, through second fluidics channel 225 and to an attached vacuum source (e.g., vacuum source 30 of FIGS. 1, 2B).

In the second configuration, fluid may also flow proximally through second fluidics channel 225, into second lumen 276 of first casing portion 260A. The fluid may flow from second lumen 276 of first casing portion 260A, into first lumen 278 of second casing portion 260B. The fluid may flow from first lumen 278 of second casing portion 260B and into first fluidics channel 224. The fluid may then flow proximally through the first lumen of an attached scope (e.g., first lumen 17 of scope 15).

Casing assembly 260 may be moved from the first configuration to the second configuration (or vice versa) by rotating first casing portion 260A relative to second casing portion 260B (or vice versa). For example, first casing portion 260A may be rotated in a first direction (e.g., clockwise or counterclockwise) and/or second casing portion 260B may be rotated in a second, opposite direction (e.g., counterclockwise or clockwise). In some aspects, casing assembly may include an indexing mechanism to facilitate alignment of first casing portion 260A with second casing portion 260B. The indexing mechanism may include mating features of the first and second portions 260A, 260B of casing assembly 260, e.g., which may provide tactile feedback to the user. For example, first casing portion 260A may include a projection receivable within an aperture of second casing portion 260B or vice versa. In some examples, proximal face 283 of first casing portion 260A may include one or more projections 287 (e.g., protrusion(s), bump(s), etc.), and distal face 285 of second casing portion 260B may include one or more apertures 289 (e.g., indentation(s), depression(s), etc.), to receive projection(s) 287 of first casing portion 260A. In aspects, projection(s) 287 and aperture(s) 289 may provide tactile feedback to the user that the first casing portion 260A and second casing portion 260B are in the first configuration and/or the second configuration (e.g., the first and second casing portions 260A, 260B are properly aligned with each other). Casing assembly 260 may also include other markers or indicators (e.g., arrows, text, etc.) on an outer surface of first casing portion 260A and/or second casing portion 260B to indicate if fluidics module 250 is in the first or second configuration.

In some aspects, casing assembly 260 may include a fastener 299. Fastener 299 may be a Velcro strap, a strap with a buckle, or any similar fastener configured for securing fluidics module 250 to a component of system 10. For example, fastener 299 may be configured to extend at least partially around handle 104 and/or shaft 106 of scope 115 (FIG. 3) to secure fluidics module 250 to scope 115. In other aspects, fastener 299 may be secured to vacuum source 30, fluid source 40, and/or to other components within a surgical suite.

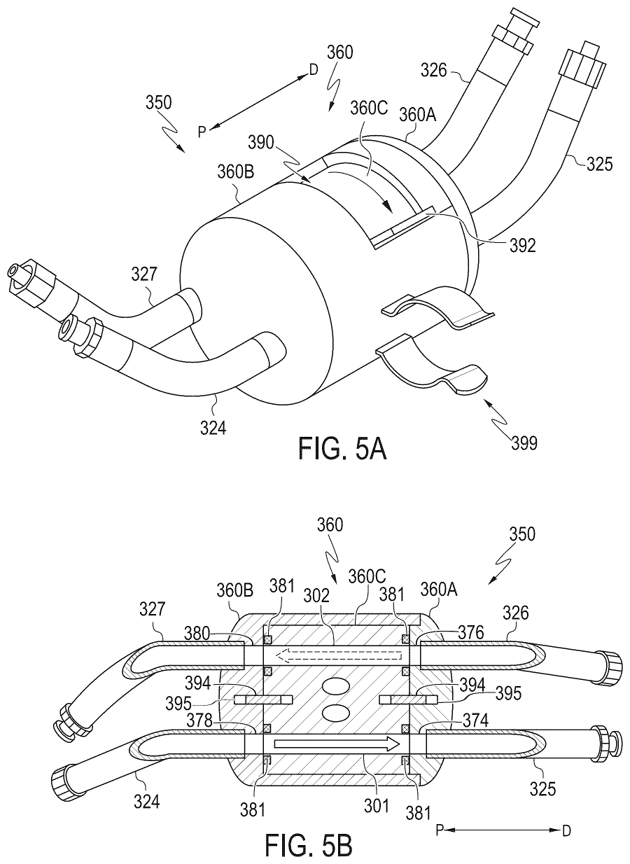

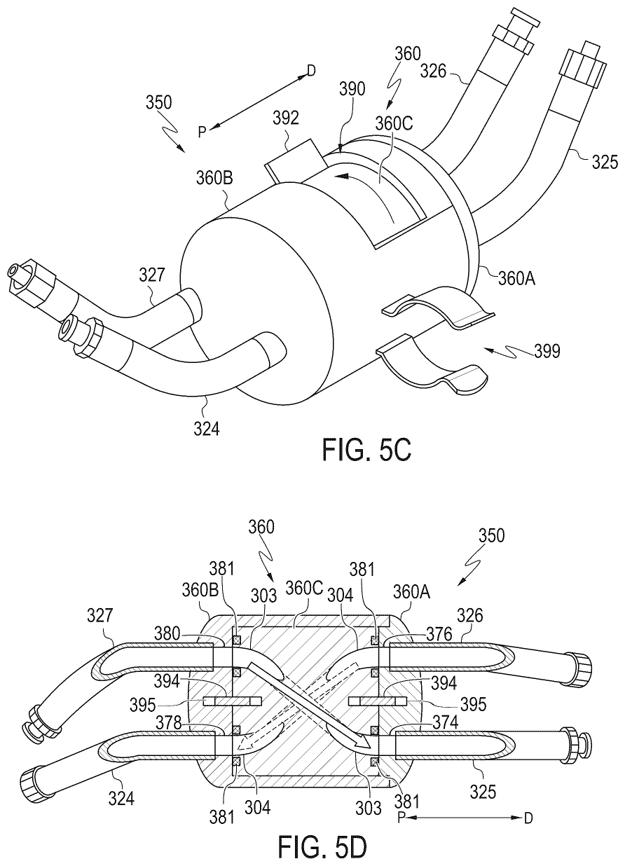

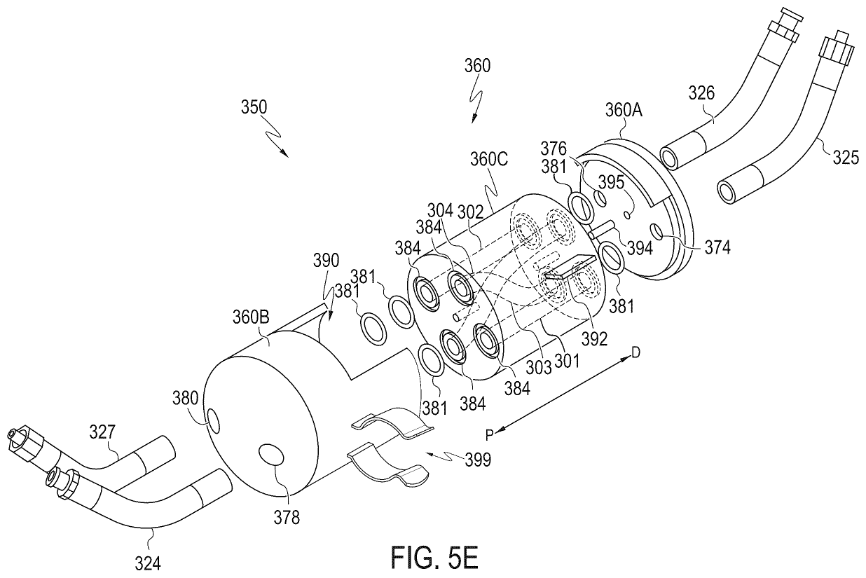

FIGS. 5A-5E illustrate another exemplary fluidics module 350 according to the present disclosure. For example, FIG. 5A illustrates a perspective view of fluidics module 350 in a first configuration; FIG. 5B illustrates a cross-sectional view of fluidics module 350 in the first configuration; FIG. 5C illustrates a perspective view of fluidics module 350 in a second configuration; FIG. 5D illustrates a cross-sectional view of fluidics module 350 in the second configuration; and FIG. 5E illustrates an exploded view of fluidics module 350. Fluidics module 350 may have any or all of the characteristics of fluidics module 250, except as described below. For example, fluidics module 350 may be utilized with system 10, shown in FIG. 1. Fluidics module 350 may be configured to switch a direction of fluid flow within first lumen 17 and second lumen 19 of scope 115 of FIG. 3.

Fluidics module 350 may include a casing assembly 360. Casing assembly 360 may include a first casing 360A permanently or removably coupled to, e.g., fixed to, a second casing 360B. First casing 360A may be coupled to second casing 360B via one or more fastening elements, e.g., mechanical fasteners, and/or an adhesive. With first casing 360A fixed to second casing 360B, casing assembly 360 may resemble a cylindrical canister. First casing 360A and second casing 360B together may define a window 390. Window 390 may be at least partially defined by one or more cutouts of second casing 360B and/or one or more walls or protrusions of first casing 360A. A manifold 360C may be rotatably disposed between first casing 360A and second casing 360B. In some aspects, the manifold 360C has a generally cylindrical shape. For example, first casing 360A and second casing 360B may at least partially surround and enclose manifold 360C. An actuator, e.g., lever 392, may extend outward from a surface of manifold 360C, for example, through window 390 of casing assembly 360. A user may engage lever 392 to rotate manifold 360C within casing assembly 360, for example, from a first end of window 390 to a second end of window 390. Rotation of manifold 360C may change a direction of fluid flowing within fluidics module 350, e.g., to thereby change a direction of fluid flow within lumens of an attached scope. Shown more clearly in FIGS. 5B and 5D, manifold 360C may rotate about one or more pins 394 fixed to opposite ends of manifold 360C. Each of first casing 360A, second casing 360B, and manifold 360C may include one or more cavities 395 to receive pins 394.

A first fluidics channel 324 may extend from a proximal end of second casing 360B. For example, second casing 360B may include a first lumen 278 to permit fluid communication between first fluidics channel 324 and one or more lumens of manifold 360C. Second fluidics channel 325 may extend from a distal end of first casing 360A. Second fluidics channel 325 may be in fluid communication with a first lumen 374 of first casing 360A. Shown more clearly in FIG. 5B, in the first configuration, first lumen 374 of first casing 360A and first lumen 378 of second casing 360B may be aligned and in fluid communication with a first lumen 301 of manifold 360C.

A third fluidics channel 326 may extend from a distal end of first casing 360A. Third fluidics channel 326 may be in fluid communication with a second lumen 376 of first casing 360A. A fourth fluidics channel 327 may extend from a distal end of second casing 360B. Fourth fluidics channel 327 may be in fluid communication with a second lumen 380 formed within second casing 360B. In the first configuration, second lumen 376 of first casing 360A and second lumen 380 of second casing 360B may be aligned and in fluid communication with a second lumen 302 of manifold 360C. First lumen 301 and second lumen 302 of manifold 360C may extend longitudinally through manifold 360C. In aspects, first lumen 301 and second lumen 302 of manifold 360C may be parallel to a longitudinal axis of manifold 360C, e.g., parallel to one another.

In the first configuration, fluid may flow from a first lumen of a connected scope (e.g., first lumen 17 of scope 15), through first fluidics channel 324. The fluid may flow through first lumen 378 of second casing 360B and into first lumen 301 of manifold 360C. The fluid may flow from first lumen 301 of manifold 360C, through first lumen 374 of first casing 360A, and through second fluidics channel 325. The fluid may flow through second fluidics channel 325 and into a reservoir of a vacuum source (e.g., vacuum source 30 of FIGS. 1, 2A). In the first configuration, fluid may also flow from a fluid source coupled to third fluidics channel 326. The fluid may flow through third fluidics channel 326, through second lumen 376 of first casing 360A. The fluid may then flow through second lumen 302 of manifold 360C, through second lumen 380 of second casing 360B, and through fourth fluidics channel 327. The fluid may flow from fourth fluidics channel 327 to a second lumen of a connected scope (e.g., lumen 19 of scope 15, shown in FIGS. 1, 2A).

In the second configuration, lever 392 may be used to rotate manifold 360C about pins 394 and within casing assembly 360. In the second configuration, a direction of fluid flow within first fluidics channel 324 and fourth fluidics channel 327 may be reversed. For example, in the second configuration, fluid may flow from the second lumen of an attached scope (e.g., second lumen 19 of scope 15 of FIGS. 1, 2B) and through fourth fluidics channel 327. The fluid may flow from fourth fluidics channel 327 and through second lumen 380 of second casing 360B. The fluid may flow from second lumen 380 of second casing 360B any through a third lumen 303 of manifold 360C. Third lumen 303 may be transverse to a longitudinal axis of manifold 360C. For example, third lumen 303 may have one or more bends and extend diagonally across manifold 360C such that third lumen 303 of manifold 360C is in fluid communication with first lumen 374 of first casing 360A. For example, third lumen 303 may form a continuous fluid path between fourth fluidics channel 327 and second fluidics channel 325 such that fourth fluidics channel 327 is in fluid communication with a vacuum source (e.g., vacuum source 30) coupled to second fluidics channel 325.

In the second configuration, fluid may flow from third fluidics channel 326, through second lumen 376 of first casing 360A, and through a fourth lumen 304 of manifold 360C. Fluid may flow through fourth lumen 304 and through first lumen 378 of second casing 360B. Fluid may continue to flow from first lumen 378 and through first fluidics channel 324. In these aspects, fourth lumen 304 of manifold 360C may form a continuous fluid path between third fluidics channel 326 and first fluidics channel 324. For example, fourth lumen 304 of manifold 360C may be transverse to a longitudinal axis of manifold 360C. For example, fourth lumen 304 may have one or more bends and extend diagonally across manifold 360C.

Sealing members 381 may be disposed within grooves 384 surrounding the opening of each lumen 301, 302, 303, 304 on each end of manifold 360C. Sealing members 381 may create a fluid-tight seal between respective lumens of first casing 360A and second 360B and lumens 301, 302, 303, 304 of manifold 360C.

Manifold 360C may be formed as a machined component, a single molded component, or a 3D-printed component. In these aspects, lumens 301, 302, 303, 304 of manifold 360C may be formed by the material comprising manifold 360C. In other aspects, lumens 301, 302, 303, 304 of manifold 360C may be formed using flexible tubes within manifold, e.g., that have been fixed within manifold 360C. For example, flexible tubes may be positioned within a mold cavity, and a material may be poured around the flexible tubes. The material may be cured so as to fix the flexible tubes in position, thus forming manifold 360C having lumens 301, 302, 303, 304. The manifold 360C may be removed from the mold cavity and assembled between first casing 360A and second casing 360B.

In some aspects, casing assembly 360 may include a fastener 399. Fastener 399 may be a clamping fastener having two bendable arms to receive a portion of a scope (e.g., scope 15 of FIGS. 1, 2A, 2B, or handle 104 and/or shaft 106 of scope 115 of FIG. 3). In these aspects, fluidics module 350 may be secured to the scope. In other aspects, fastener 399 may be secured to vacuum source 30, fluid source 40, and/or other components within a surgical suite. In other aspects, casing assembly 360 may include a quick-release button, for example, to enable a user to transition from the first configuration to the second configuration.

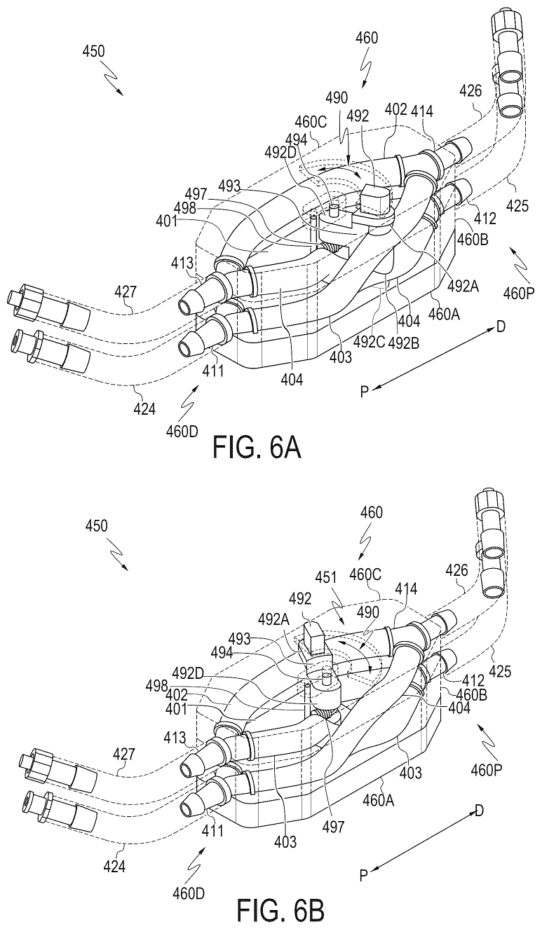

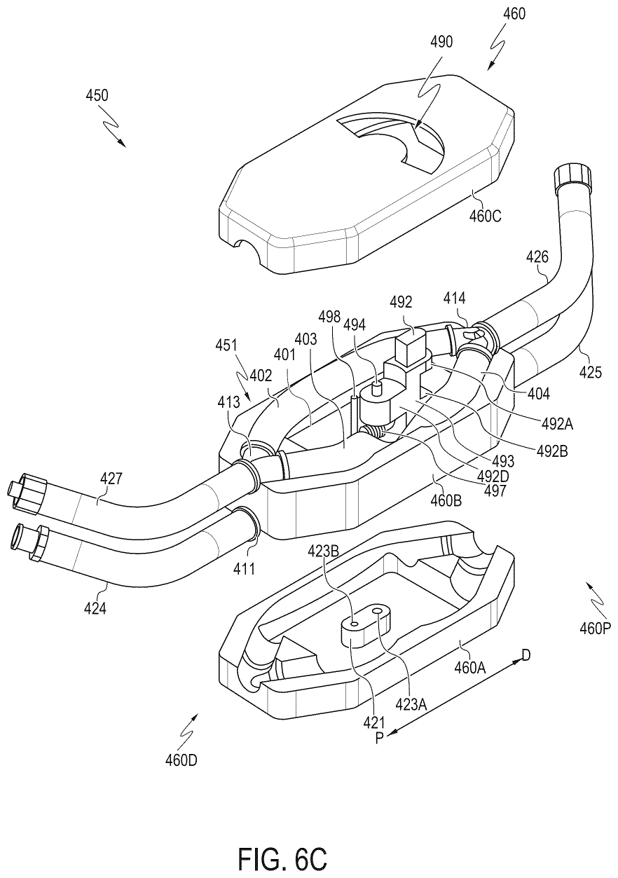

FIGS. 6A-6C illustrate another exemplary fluidics module 450 according to the present disclosure. For example, FIG. 6A illustrates fluidics module 450 in a first configuration; FIG. 6B illustrates fluidics module 450 in a second configuration; and FIG. 6C illustrates an exploded view of fluidics module 450. Aspects of fluidics module 450 are shown in broken lines in FIGS. 6A and 6B to assist in illustrating internal components of fluidics module 450. Fluidics module 450 may have any or all of the characteristics of fluidics modules 250 and/or 350, except as described below. For example, fluidics module 450 may be utilized with system 10, shown in FIG. 1. Fluidics module 450 may be configured to switch a direction of fluid flow within first lumen 17 and second lumen 19 of scope 15 of FIGS. 1, 2A, 2B.

Fluidics module 450 may include a casing assembly 460. Casing assembly 460 may include a base 460A, a center portion 460B, and a cover, or top portion, 460C. In some aspects, center portion 460B may be integrally formed with base 460A. Cover 460C may be fixed to center portion 460B, for example, on an opposite side of base 460A. Center portion 460B may be coupled to base 460A and cover 460C may be coupled to, e.g., fixed to, center portion 460B by one or more fastening elements, such as mechanical fasteners, and/or an adhesive. Together, base 460A, center portion 460B, and cover 460C may form an enclosure to contain a plurality of lumens 451 and an actuator 492. Cover 460C may include a window 490, through which a portion of an actuator 492 may at least partially extend from.

A plurality of lumens 451 may extend longitudinally between a proximal end 460P and a distal end 460D of casing assembly 460. For example, a first lumen 401 may extend from a first Y-connector 411 at a first, proximal end 460P of casing assembly 460 to a second Y-connector 412 at a second, distal end 460D of casing assembly 460. In aspects, first lumen 401 may extend through casing assembly 460, parallel to a longitudinal axis of casing assembly 460 and in a first plane. First Y-connector 411, second Y-connector 412 and first lumen 401 may all be in the same first plane.

A second lumen 402 may extend through casing assembly 460, for example, in a second plane that is parallel to the longitudinal axis of casing assembly 460. Second lumen 402 may extend from a third Y-connector 413 at a distal end 460D of casing assembly 460 to a fourth Y-connector 414 at a proximal end 460P of casing assembly 460. Third Y-connector 413, fourth Y-connector 414 and second lumen 402 may all be in the same second plane. The first plane may be below the second plane.

A third lumen 403 may extend from first Y-connector 411 at the distal end 460D of casing assembly 460 to fourth Y-connector 414 at the proximal end 460P of casing assembly 460. Third lumen 403 may extend diagonally through casing assembly 460. For example, third lumen 403 may extend from first Y-connector 411 at a bottom left corner of casing assembly 460 to fourth Y-connector 414 at a top right corner of casing assembly 460. First Y-connector 411 may be in the first plane, fourth Y-connector 414 may be in the second plane, and third lumen 403 may extend diagonally between the first plane and the second plane.

A fourth lumen 404 may extend from third Y-connector 413 of the distal end 460D of casing assembly 460 to second Y-connector 412 at the proximal end 460P of casing assembly 460. Fourth lumen 404 may extend diagonally through casing assembly 460. For example, fourth lumen 404 may extend from third Y-connector 413 at a top left corner of casing assembly 460 to second Y-connector 412 at a bottom right corner of casing assembly 460. Second Y-connector 412 may be in the first plane, third Y-connector 413 may be in the second plane, and fourth lumen 404 may extend diagonally between the second plane and the first plane.

First Y-connector 411 may be fluidly coupled to a first fluidics channel 424. First fluidics channel 424 may be fluidly coupled to a vacuum source (e.g., vacuum source 30 of FIGS. 1, 2A, 2B). Second Y-connector may be fluidly coupled to a second fluidics channel 425. Second fluidics channel 425 may be fluidly coupled to a scope (e.g., scope 15 of FIGS. 1, 2A, 2B or scope 115 of FIG. 3). Third Y-connector 413 may be fluidly coupled to a fourth fluidics channel 427. Fourth fluidics channel 427 may be fluidly coupled to the scope (e.g., scope 15 of FIGS. 1, 2A, 2B or scope 115 of FIG. 3). Fourth Y-connector 414 may be fluidly coupled to third fluidics channel 426. Third fluidics channel 426 may be fluidly coupled to a fluid source (e.g., fluid source 40 of FIGS. 1, 2A, 2B).

Actuator 492 may be at least partially disposed within casing assembly 460. Actuator 492 may include a first arm 492A, a second arm 492B and a third arm 492C extending laterally in a first direction, for example, away from a stem portion 493. A fourth arm 492D may extend laterally away from stem portion 493 in a second direction (e.g., opposite the first direction). Fourth arm 492D may include a pin 494 protruding perpendicularly from a top surface of fourth arm 492D. A similar pin may protrude perpendicularly from a bottom surface of fourth arm 492D. Actuator 492 may be configured to rotate about pin 494. Base 460A and/or cover 460C may include features to contain a similar pin protruding from the bottom surface of fourth arm 492D. For example, shown more clearly in FIG. 6C, base 460A may include a protrusion 421 having a first opening 423A configured to at least partially receive a pin protruding from the bottom surface of fourth arm 492D. Cover 460C may include similar features to at least partially receive pin 494 protruding from the top surface of fourth arm 492D. In these aspects, the features of base 460A and cover 460C may receive pin 494 while still permitting rotation of actuator 492.

Actuator 492 may be configured to receive a lumen (e.g., one of second lumen 402 or third lumen 403) between first arm 492A and second arm 492B and a lumen (e.g., one of first lumen 401 and fourth lumen 404) between second arm 492B and third arm 492C. For example, in the first configuration, shown in FIG. 6A, third lumen 403 is received between first arm 492A and second arm 492B and fourth lumen 404 is received between second arm 492B and third arm 492C. Actuator 492 may thus pinch off or prevent fluid flow within each of third lumen 403 and fourth lumen 404. For example, actuator 492 may be configured to stop or prevent fluid flow within third lumen 403 and fourth lumen 404 while permitting fluid flow within first lumen 401 and second lumen 402 (i.e., the lumens that are not received between the arms of actuator 492). To prevent fluid flow within third lumen 403 and fourth lumen 404 may be pinched off or closed between respective arms of actuator 492. In this configuration, fluid may flow between first fluidics channel 424 and second fluidics channel 425 via first lumen 401 of fluidics module 450, and between third fluidics channel 426 and fourth fluidics channel 427 via second lumen 402 of fluidics module 450.

In the second configuration, actuator 492 may be rotated about pins 494 to a second position shown in FIG. 6A. In the second configuration, second lumen 402 is received between first arm 492A and second arm 492B and first lumen 401 is received between second arm 492B and third arm 492C. In these aspects first lumen 401 and second lumen 402 may be pinched off, or closed, thus preventing fluid flow through first lumen 401 and second lumen 402. Fluid flow may be permitted through third lumen 403 and fourth lumen 404. In these aspects, fluid may flow between first fluidics channel 424 and third fluidics channel 426 via fourth lumen 404 of fluidics module 450, and between second fluidics channel 425 and fourth fluidics channel 427 via third lumen 403 of fluidics module 450.

Fluidics module 450 may further include a biasing member 497 (e.g., a coiled spring or similar). A first end of biasing member 497 may be fixed to a portion of actuator 492 (e.g., under fourth arm 492D, and a second end of biasing member 497 may be fixed to a fixed shaft 498. Fixed shaft 498 may extend between base 460A and cover 460C. In some aspects, fixed shaft 498 may be at least partially received within a second cavity 423B of protrusion 421 on base 460A and/or within a similar cavity on cover 460C. Biasing member 497 may be configured to bias actuator 492 in the first configuration or the second configuration.

Any of fluidics modules 250, 350, 450 may include one or more sensors, for example, to determine a direction of fluid flow within the lumens of the fluidics module and/or measure pressure, temperature, speed, or any other aspect of the fluid flowing within the fluidics module.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a casing assembly including a first portion movably coupled to a second portion;
a first fluidics channel extending from a first end of the casing assembly that includes the first portion;
a second fluidics channel extending from a second end of the casing assembly that includes the second portion;
a third fluidics channel extending from the second end of the casing assembly; and
a fourth fluidics channel extending from the first end of the casing assembly,
wherein moving the first portion relative to the second portion transitions the medical device between a first configuration and a second configuration;
wherein, in the first configuration, the first fluidics channel and the second fluidics channel are fluidly connected, and the third fluidics channel and the fourth fluidics channel are fluidly connected, and
wherein, in the second configuration, the first fluidics channel and the third fluidics channel are fluidly connected, and the second fluidics channel and the fourth fluidics channel are fluidly connected.

2. The medical device of claim 1, wherein, in the first configuration, the first and second fluidics channels are in communication and configured to provide fluid flow in a first direction through the casing assembly, and the third and fourth fluidics channels are in communication and configured to provide fluid flow in a second direction through the casing assembly, opposite the first direction.

3. The medical device of claim 1, wherein the first portion defines a first lumen and a second lumen, and the second portion defines a third lumen and a fourth lumen.

4. The medical device of claim 3, wherein:
in the first configuration, the first lumen is fluidly connected to the third lumen, and the second lumen is fluidly connected to the fourth lumen, and
in the second configuration, the first lumen is fluidly connected to the fourth lumen, and the second lumen is fluidly connected to the third lumen.

5. The medical device of claim 1, wherein the first portion is rotatable relative to the second portion.

6. The medical device of claim 1, wherein the casing assembly includes a manifold disposed between the first portion and the second portion, wherein the manifold includes a first lumen, a second lumen, a third lumen, and a fourth lumen extending therethrough.

7. The medical device of claim 6, wherein the second lumen of the manifold and the fourth lumen of the manifold are transverse to a longitudinal axis of the manifold.

8. The medical device of claim 7, wherein the first lumen of the manifold and the second lumen of the manifold are parallel to the longitudinal axis of the manifold.

9. The medical device of claim 6, wherein:
in the first configuration, the first fluidics channel and the second fluidics channel are fluidly connected via the first lumen of the manifold, and the third fluidics channel and fourth fluidics channel are fluidly connected via the second lumen of the manifold; and
in the second configuration, the first fluidics channel and the third fluidics channel are fluidly connected via the third lumen of the manifold, and the second fluidics channel and the fourth fluidics channel are fluidly connected via the fourth lumen of the manifold.

10. The medical device of claim 6, wherein the casing assembly includes an actuator movable within a window formed by the first portion and the second portion of the casing assembly to move the medical device between the first configuration and the second configuration.

11. The medical device of claim 1, further comprising a plurality of sealing members configured to provide a fluid-tight seal around each respective first, second, third, and fourth fluidics channels.

12. The medical device of claim 1, wherein one of the first portion or the second portion includes an indexing mechanism.

13. The medical device of claim 12, wherein the indexing mechanism includes a projection receivable within an aperture, one of the first portion or the second portion including the projection and the other of the first portion or the second portion including the aperture.

14. The medical device of claim 1, wherein the first fluidics channel and the fourth fluidics channel are configured to be fluidly coupled to a scope.

15. The medical device of claim 1, wherein the casing assembly includes a fastener configured to couple the medical device to a scope.

16. A medical device comprising:
a casing assembly;
a first fluidics channel extending from a first end of the casing assembly;
a second fluidics channel extending from a second end of the casing assembly;
a third fluidics channel extending from the second end of the casing assembly; and
a fourth fluidics channel extending from the first end of the casing assembly;
wherein the casing assembly includes an actuator to transition the medical device between a first configuration and a second configuration;
wherein, in the first configuration, the first fluidics channel and the second fluidics channel are fluidly connected, and the third fluidics channel and the fourth fluidics channel are fluidly connected; and
wherein, in the second configuration, the first fluidics channel and the third fluidics channel are fluidly connected, and the second fluidics channel and the fourth fluidics channel are fluidly connected.

17. The medical device of claim 16, wherein the casing assembly includes a plurality of Y-connectors.

18. The medical device of claim 16, wherein the actuator is movable within a window of the casing assembly to move the medical device between the first configuration and the second configuration.

19. A medical device comprising:
a casing assembly including a first portion, a second portion, and a manifold between the first portion and the second portion;
a first fluidics channel extending from a first end of the casing assembly that includes the first portion;
a second fluidics channel extending from a second end of the casing assembly that includes the second portion;
a third fluidics channel extending from the second end of the casing assembly; and
a fourth fluidics channel extending from the first end of the casing assembly;
wherein the manifold includes a first lumen, a second lumen, a third lumen, and a fourth lumen extending therethrough;
wherein, in a first configuration of the medical device, the first fluidics channel and the second fluidics channel are fluidly connected via the first lumen, and the third fluidics channel and the fourth fluidics channel are fluidly connected via the second lumen, and
wherein, in a second configuration of the medical device, the first fluidics channel and the third fluidics channel are fluidly connected via the third lumen, and the second fluidics channel and the fourth fluidics channel are fluidly connected via the fourth lumen.

20. The medical device of claim 19, wherein the casing assembly includes an actuator configured to transition the medical device between the first configuration and the second configuration.
